(19) United States
(12) Patent Application Publication
(10) Pub. No.: US 2022/0023085 A1
(43) Pub. Date: Jan. 27, 2022

(54) HUNCHBACK ORTHOSIS

(71) Applicant: WELLPOWER COMMERCE HOLDING CO., LIMITED, Hong Kong (CN)

(72) Inventor: SHING-JIU SHEU, HONG KONG (CN)

(21) Appl. No.: 16/934,370

(22) Filed: Jul. 21, 2020

(51) Int. Cl. A61F 5/02

(52) U.S. Cl. CPC A61F 5/026; A61M 2021/0022

(57) ABSTRACT

The invention provides a wearing assembly for wearing on a user's shoulders, and a reminder device for generating vibrations or sounds to remind the user to adjust his/her current body posture. When the user is in a hunchback posture, the width of the user's back increases to exert enough outward pulling force on the reminder device and triggers vibrations or sounds. The wearing assembly includes two connecting belts and two shoulder wear pieces. The reminder device includes a casing, a fixed fastener, a movable fastener, a first guide member, a second guide member, a first contact end, a counting module, and a microswitch. When the first contact end moves and contacts the microswitch, the microswitch will enter a conductive state, so that the casing will produce vibrations or sounds. The counting module is used to accumulate the number of times the microswitch is contacted within a period of time.

### BACKGROUND OF THE INVENTION

### 1. Field of the Invention

The present invention relates to a posture-correcting orthosis, especially a hunchback orthosis.

### 2. Description of the Related Art

Thoracic kyphosis may be congenital or acquired. The acquired thoracic kyphosis is often caused by unhealthy postures such as unhealthy way of sitting or uneven muscle training resulting in imbalanced muscle tension, decreased physical perception, and declined self-correction ability whereby appearance of hunchback emerges. Nowadays, hunchbacks often appear in groups such as students, sedentary office workers, and vehicle drivers. These groups of people often have their thoracic spine bent for prolonged periods of time.

At present, the hunchback orthoses are very popular. Please refer to FIG. 5 for a commercially available hunchback orthosis that uses a body bending angle to trigger vibrations. For example, when the body bending angle exceeds 25 degrees, a vibration is triggered to remind the user to adjust his/her posture. Please refer to FIG. 6 for another commercially available hunchback orthosis using elastic bands to strongly pull the user's back to correct the posture.

However, the above-mentioned hunchback orthosis that uses the bending angle of the body to trigger vibrations is less sensitive and often cannot accurately remind the user to adjust his/her postures. The above-mentioned hunchback orthosis that uses elastic bands to strongly pull the back tends to make the user feel uncomfortable due to significant tensions exerted from the elastic bands on the user's body. Therefore, the existing hunchback orthoses must be further improved.

### SUMMARY OF THE INVENTION

The present invention relates to a hunchback orthosis. The present invention provides an adjustable wearing assembly for wearing on the shoulders of a user and a reminder device for generating a vibration or a sound to remind the user to adjust his/her current body posture to overcome problems of the prior art. The technical details of the hunchback orthosis of the present invention includes the following:

a wearing assembly for wearing on shoulders of a user, including:

two connecting belts, wherein when the user wears the wearing assembly on the shoulders, the two connecting belts are disposed along a direction which is perpendicular to the user's spine;

two shoulder wear pieces respectively connected to the two connecting belts, and the two shoulder wear pieces being for fastening on the user's shoulders;

a reminder device for generating a vibration or a sound to remind the user to adjust a current body posture of the user, including:

a casing;

a fixed fastener located on one side of the casing and connected to one of the two connecting belts which pulls the fixed fastener without causing any relative movement between the fixed fastener and the casing;

a movable fastener located on an opposite side of the casing and connected to another of the two connecting belts which pulls and accordingly moves the movable fastener relative to the casing;

a first guide member fixed on one end of the movable fastener and passing through a side wall of the casing;

a second guide member fixed to another end of the movable fastener, and passing through the side wall of the casing;

a first contact end, fixed to one end of the first guide, and moving along following the movement of the movable fastener;

a microswitch, fixed in the casing, wherein when the first contact end moves and contacts the microswitch, the microswitch enters a conductive state, so that the casing will produce the vibration or the sound, and when the first contact end no longer contacts the microswitch, the microswitch enters an off state, so that the casing stops producing the vibration or the sound;

a counting module, fixed in the casing, and used to accumulate the number of times the microswitch is contacted and enters the conductive state within a period of time.

The reminder device further includes:

a vibration motor, fixed in the casing, and used for generating the vibration;

a speaker, fixed in the casing, and used for generating the sound;

a battery, fixed in the casing, and used for providing a power source; wherein

the counting module, the vibration motor, and the speaker are connected in parallel to form a functional circuit, and the functional circuit is connected in series with the microswitch and the battery.

The wearing assembly further includes:

two first adjusting devices respectively combined with the two shoulder wear pieces to form two rings, each of the two first adjusting devices used to adjust the size of the respective ring to properly fasten to the user's shoulders;

two second adjusting devices respectively combined with the two connecting belts to adjust the length of the connecting belt to match the user's shoulder width.

The reminder device further includes:

a first elastic return member, fixed in the casing, and used for contacting and resisting the first contact end; wherein

when there is no relative displacement between the movable fastener and the casing, the movable fastener, the first guide member, and the first contact end are all in their initial positions;

when the first guide member moves outward with the movable fastener and leaves its respective initial position, the first elastic return member is pressed to generate elastic forces resisting the outward movement;

when an outward pulling force gradually subsides, the first elastic return member gradually pushes the movable fastener, the first guide, and the first contact end, back to their initial positions.

When the movable fastener is moved by being pulled by another of the two connecting belts, and the movement exceeds a displacement threshold value, the first contact end will contact the microswitch, causing the microswitch to enter the conductive state.

When the movable fastener is moved by being pulled by another of the two connecting belts, and the movement is less than a displacement threshold value, the first contact end and the microswitch will be separated, causing the microswitch to enter the off state.

The counting module is further equipped with a display device, and the display device is exposed through an opening or a transparent area on the surface of the casing so that the user can perceive information displayed by the display device.

The first elastic return member is a spring.

Each of the two first adjusting devices is an adjusting buckle, a hook and loop fastener, a snap or a magnetic connector.

Each of the two second adjusting devices is a hook and loop fastener, an adjusting buckle, a snap or a magnetic connector.

### BRIEF DESCRIPTION OF THE DRAWINGS

FIG. 1 is a perspective view of the hunchback orthosis of the present invention;

FIG. 2 is a schematic structural diagram of a reminder device in the hunchback orthosis of the present invention;

FIG. 3 is a circuit block diagram of the reminder device in the hunchback orthosis of the present invention;

FIG. 4 is an operational view of the hunchback orthosis of the present invention;

FIG. 5 is a prior art that uses a body bending angle to trigger vibrations;

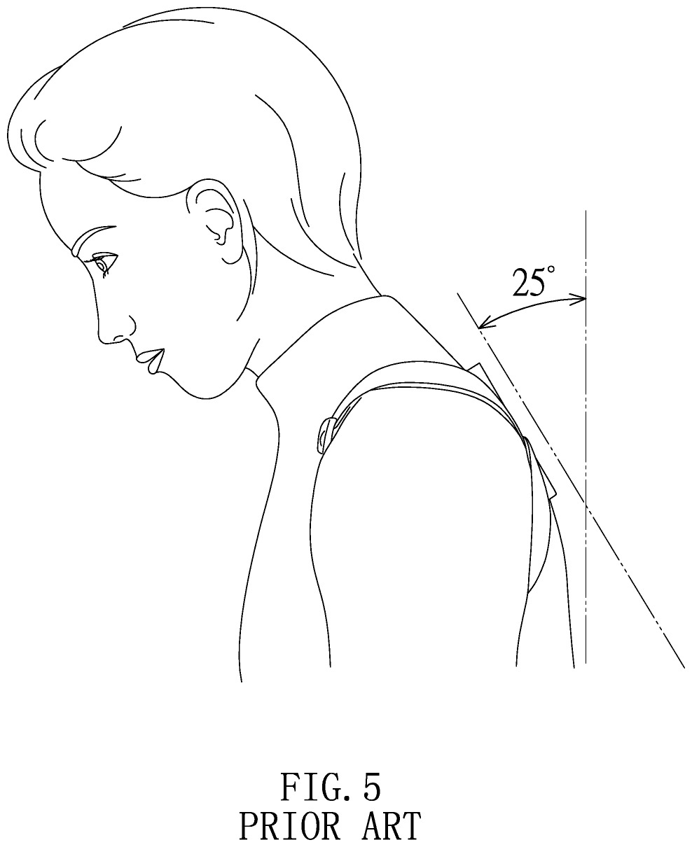

FIG. 6 is another prior art that uses elastic bands to pull a user's back.

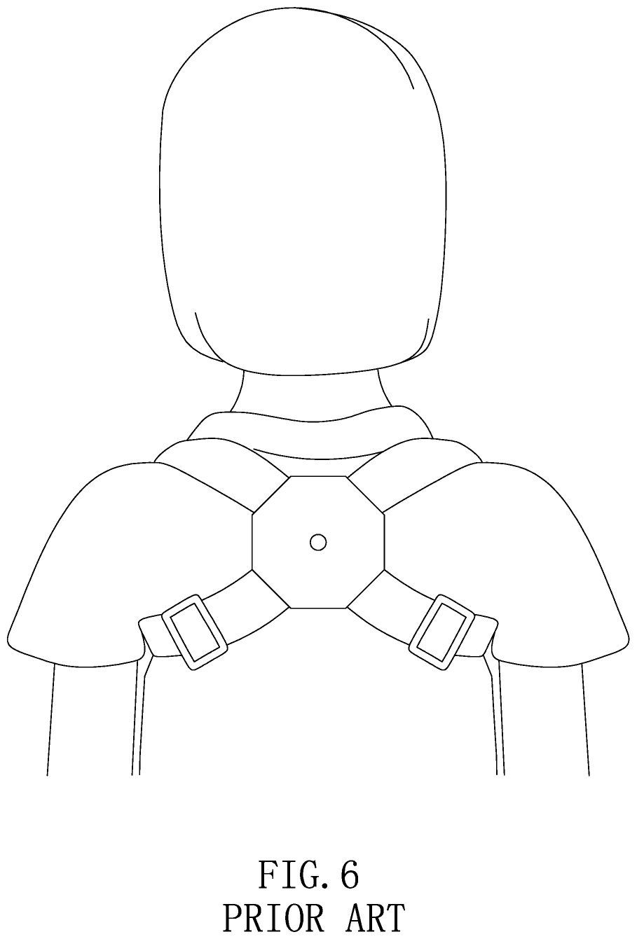

### DETAILED DESCRIPTION OF THE INVENTION

In the following, the technical solutions in the embodiments of the present invention will be clearly and fully described with reference to the drawings in the embodiments of the present invention. Obviously, the described embodiments are only a part of the embodiments of the present invention, but not all the embodiments. Based on the embodiments of the present invention, all other embodiments obtained by a person of ordinary skill in the art without creative efforts shall fall within the protection scope of the present invention.

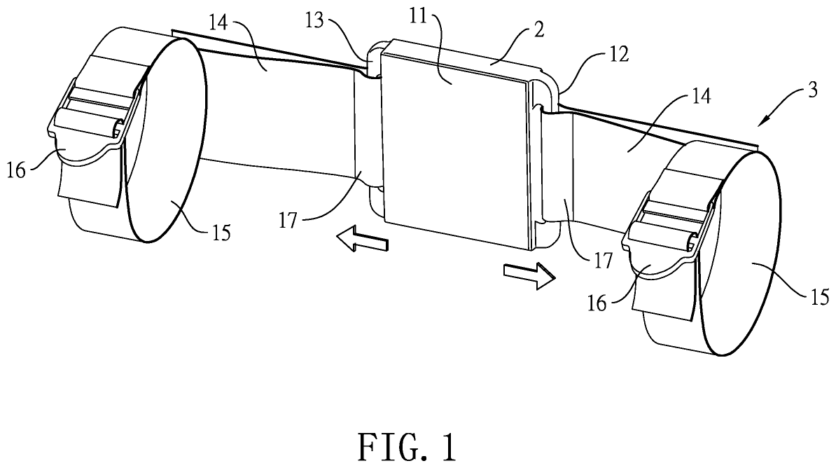

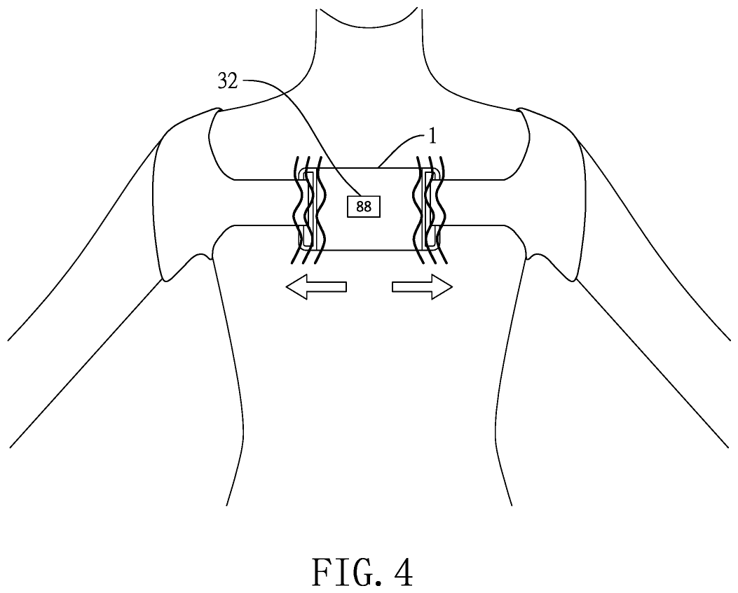

Please refer to FIG. 1 and FIG. 4. FIG. 1 is a perspective view of the hunchback orthosis of the present invention and FIG. 4 is an operational view of the hunchback orthosis of the present invention. A hunchback orthosis 1 includes: a reminder device 2 and a wearing assembly 3. The reminder device 2 is for generating sounds and vibrations to notify a user that the reminder device 2 has been triggered to remind the user to adjust his/her current body posture to a straight posture. The wearing assembly 3 is for facilitating the user to wear the hunchback orthosis 1 on the user's shoulders. The reminder device 2 has a casing 11, and at both ends of the casing 11, a movable fastener 12 and a fixed fastener 13 are respectively provided. The movable fastener 12 will be displaced relative to the casing 11 as a result of the outward pulling force from the wearing assembly 3 (as indicated by the arrow in FIG. 1 and FIG. 4). However, the fixed fastener 13 will not be displaced relative to the casing 11 despite the outward pulling force exerted from the wearing assembly 3 (as indicated by the arrow in FIG. 1 and FIG. 4).

The wearing assembly 3 has two similar portions symmetrically separated, and is respectively provided with a connecting belt 14, a shoulder wear piece 15, and a first adjusting device 16 such as an adjusting buckle, a hook and loop fastener, a snap or a magnetic connector, and a second adjusting device 17 such as a hook and loop fastener, an adjusting buckle, a snap or a magnetic connector. The movable fastener 12 and the fixed fastener 13 are respectively fixedly connected to one end of the corresponding connecting belt 14, and the connecting belt 14 is further combined with the second adjusting device 17 to adjust the length of the connecting belt 14 to match the shoulder width of the user. The other end of the connecting belt 14 is fixedly connected to the shoulder wear piece 15 and the shoulder wear piece 15 is combined with the first adjusting device 16 to form a ring. The first adjusting device 16 can be used to adjust the size of the ring to properly fasten to the user's shoulders. The connecting belt 14 is preferably an inelastic webbing. When the user puts on the shoulder wear piece 15, the connecting belt 14 is disposed along a direction which is perpendicular to the user's spine, and the connecting belt 14 will tend to pull the movable fastener 12 and the fixed fastener 13 outward (as shown by the arrows in FIG. 1). As the user's posture changes, the connecting belt 14 pulls the movable fastener 12 and the fixed fastener 13 with different forces.

When the user's posture is normal, although the movable fastener 12 and the fixed fastener 13 are pulled by the two portions of the wearing assembly 3, a relative displacement between the movable fastener 12 and the casing 11 will not exceed a displacement threshold value. When the user's shoulder expands, for example, the user's back relaxes and the hunchback occurs, causing the user's back to stretch to the left and right, making the user's back width increase, then, the relative displacement generated between the movable fastener 12 and the casing 11 will exceed the displacement threshold value, which in turn triggers the reminder device 2.

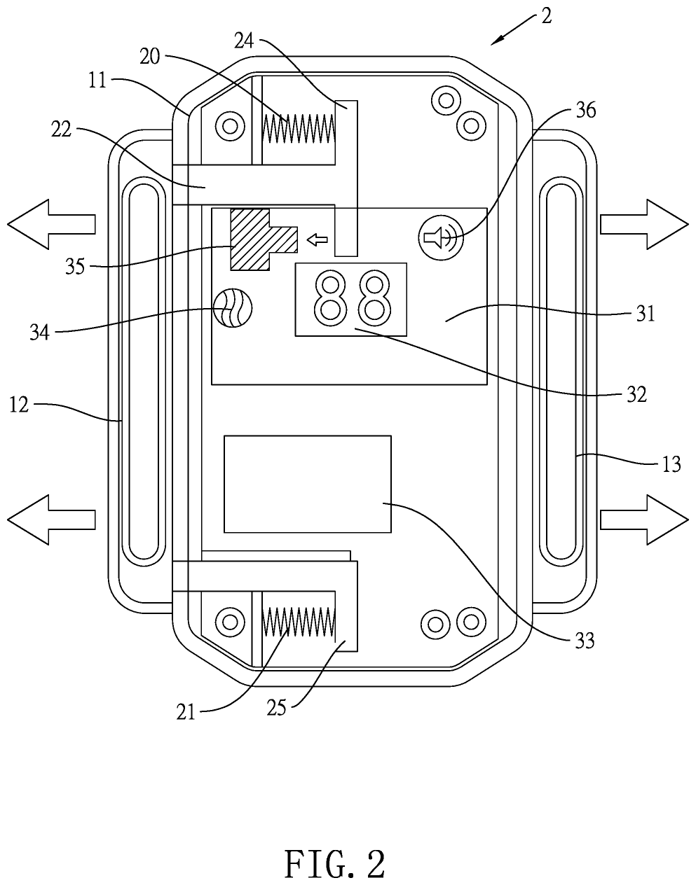

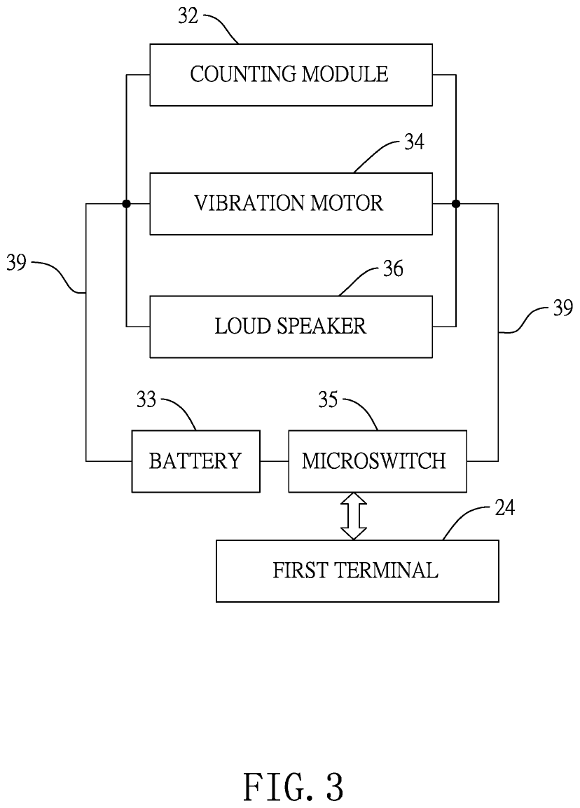

Please refer to FIG. 2 and FIG. 3. FIG. 2 is a schematic structural diagram of a reminder device in the hunchback orthosis of the present invention, and FIG. 3 is a circuit block diagram of the reminder device in the hunchback orthosis of the present invention. The reminding device 2 includes: the casing 11 in a shape of a box, the movable fastener 12 and the fixed fastener 13 respectively disposed on two opposite sides of the casing 11, a first guide member 22 and a second guide member 23 respectively disposed through a side wall of the casing 11 on the movable fastener 12, a first elastic return member 20, a second elastic return member 21, a first contact end 24, a second contact end 25, a circuit board 31, a counting module 32, a battery 33, a vibration motor 34, a microswitch 35, a speaker 36, and a wire 39.

As shown in FIG. 2, under a normal working condition, the external pulling forces on the movable fastener 12 and the fixed fastener 13 are in opposite directions, as shown by the arrow in FIG. 2. The first guide member 22 and the second guide member 23 are fixed to the two ends of the movable fastener 12 respectively. When a relative displacement between the casing 11 and the movable fastener 12 occurs as a result of the movable fastener 12 receiving outward pulling force (as shown by the arrow in FIG. 2), the first guide member 22 and the second guide member 23 will also move along following the movable fastener 12. The first contact end 24 is fixed at one end of the first guide member 22 and together forms a “T” shape, and the second contact end 25 is fixed at one end of the second guide member 23 and together forms an “L” shape, the first elastic return member 20 and the second elastic return member 21 respectively contact and resist the first contact end 24 and the second contact end 25. The first elastic return member 20 and the second elastic return member 21 are elastic objects such as return springs having a shape recovering capability.

When there is no relative displacement between the movable fastener 12 and the casing 11, the movable fastener 12, the first guide member 22, the second guide member 23, the first contact end 24, and the second contact end 25 are all in their initial positions. When the first guide member 22 and the second guide member 23 move outward with the movable fastener 12 (as shown by the arrow in FIG. 2) and leave their respective initial positions, the first elastic return member 20 and the second elastic return member 21 are pressed to generate elastic forces resisting the outward movement. When the outward pulling force gradually subsides, the first elastic return member 20 and the second elastic return member 21 will gradually push the movable fastener 12, the first guide 22, the second guide 23, the first contact end 24, and the second contact end 25 back to their initial positions.

When the user is in a hunchback posture, the width of the user's back increases, and the outward pulling force on the movable fastener 12 becomes larger (as shown by the arrow in FIG. 2), so that a relative displacement between the movable fastener 12 and the casing 11 reaches or exceeds a displacement threshold value, and the movable fastener 12 will pull the first guide member 22 and the first contact end 24 so that the first contact end 24 will contact the microswitch 35, and then the microswitch 35 is switched on to enter a conductive state. When the user's body posture is adjusted to be normal, the width of the user's back is reduced, and the outward pulling force becomes small enough to reduce the relative displacement less than the displacement threshold value again, then the first contact end 24 no longer contacts the microswitch 35, and the microswitch 35 will be switched off and enter an off state.

The counting module 32, the vibration motor 34, the microswitch 35, and the speaker 36 are disposed on the circuit board 31, as shown in FIG. 3. The counting module 32, the vibration motor 34, and the speaker are connected in parallel to form a functional circuit. And the battery 33, the microswitch 35, and the functional circuit are connected in series via the wire 39. When the microswitch 35 is in the conductive state, the battery 33 can supply power to the counting module 32, the vibration motor 34, and the speaker 36 for their normal working. When the microswitch 35 is in the off state, the battery 33 cannot supply power, and the counting module 32, the vibration motor 34, and the speaker 36 stop working.

The counting module 32 is used to accumulate the number of times that the microswitch 35 is contacted to enter the conductive state within a period of time, which is called a cumulative reminder count. The counting module 32 is further equipped with a display device, the display device is configured in such a way that the characters and images displayed by the display device can be seen from the outside of the casing 11. For example, the display device is exposed through an opening on the surface of the casing 11 or the surface of the casing 11 has a transparent area so that the user can see through it to perceive the cumulative reminder count and other images displayed by the display device. Whenever the microswitch 35 is contacted to enter the conductive state, the counting module 32 will increase the cumulative reminder count by 1 and display the cumulative reminder count on the display device, the vibration motor 34 will generate vibrations, and the speaker 36 will generate sounds to remind the user to pay attention to his/her body posture.

The aforementioned are preferred embodiments of the present invention. It should be noted that for those of ordinary skill in the art, without departing from the principles of the present invention, certain improvements and retouches of the present invention can still be made which are nevertheless considered as within the protection scope of the present invention.

Even though numerous characteristics and advantages of the present invention have been set forth in the foregoing description, together with details of the structure and function of the invention, the disclosure is illustrative only. Changes may be made in detail, especially in matters of shape, size, and arrangement of parts within the principles of the invention to the full extent indicated by the broad general meaning of the terms in which the appended claims are expressed.

What is claimed is:

1. A hunchback orthosis, including:
a wearing assembly for wearing on shoulders of a user, including:
two connecting belts, wherein when the user wears the wearing assembly on the shoulders, the two connecting belts are disposed along a direction which is perpendicular to the user's spine;
two shoulder wear pieces respectively connected to the two connecting belts, and the two shoulder wear pieces being for fastening on the user's shoulders;
a reminder device for generating a vibration or a sound to remind the user to adjust a current body posture of the user, including:
a casing;
a fixed fastener located on one side of the casing and connected to one of the two connecting belts which pulls the fixed fastener without causing any relative movement between the fixed fastener and the casing;
a movable fastener located on an opposite side of the casing and connected to another of the two connecting belts which pulls and accordingly moves the movable fastener relative to the casing;
a first guide member fixed on one end of the movable fastener and passing through a side wall of the casing;
a second guide member fixed to another end of the movable fastener, and passing through the side wall of the casing;
a first contact end, fixed to one end of the first guide member, and moving along following movement of the movable fastener; a micro switch, fixed in the casing, when the first contact end moves and contacts the microswitch, the microswitch enters a conductive state, so that the casing will produce the vibration or the sound, and when the first contact end no longer contacts the microswitch, the microswitch enters an off state, so that the casing stops producing the vibration or the sound;
a counting module, fixed in the casing, and used to accumulate the number of times the microswitch is contacted and enters the conductive state within a period of time.

2. The hunchback orthosis as claimed in claim 1, wherein the reminder device further includes:
a vibration motor, fixed in the casing, and used for generating the vibration;
a speaker, fixed in the casing and used for generating the sound;
a battery, fixed in the casing and used for providing a power source; wherein
the counting module, the vibration motor, and the speaker are connected in parallel to form a functional circuit, and the functional circuit is connected in series with the microswitch and the battery.

3. The hunchback orthosis as claimed in claim 1, wherein the wearing assembly further includes:
two first adjusting devices respectively combined with the two shoulder wear pieces to form two rings, each of the two first adjusting devices used to adjust the size of the respective ring to properly fasten to the user's shoulders.
two second adjusting devices respectively combined with the two connecting belts to adjust the length of the connecting belt to match the user's shoulder width.

4. The hunchback orthosis as claimed in claim 1, wherein the reminder device further includes:
a first elastic return member, fixed in the casing, and used for contacting and resisting the first contact end; wherein
when there is no relative displacement between the movable fastener and the casing, the movable fastener, the first guide member, and the first contact end are all in their initial positions;
when the first guide member moves outward with the movable fastener and leaves its respective initial position, the first elastic return member is pressed to generate elastic forces resisting the outward movement;
when an outward pulling force gradually subsides, the first elastic return member gradually pushes the movable fastener, the first guide, and the first contact end, back to their initial positions.

5. The hunchback orthosis as claimed in claim 1, wherein
when the movable fastener is moved by being pulled by another of the two connecting belts, and the movement exceeds a displacement threshold value, the first contact end will contact the microswitch, causing the microswitch to enter the conductive state.

6. The hunchback orthosis as claimed in claim 1, wherein
when the movable fastener is moved by being pulled by another of the two connecting belts, and the movement is less than a displacement threshold value, the first contact end and the microswitch will be separated, causing the microswitch to enter the off state.

7. The hunchback orthosis as claimed in claim 1, wherein
the counting module is further equipped with a display device, and the display device is exposed through an opening or a transparent area on the surface of the casing so that the user can perceive information displayed by the display device.

8. The hunchback orthosis as claimed in claim 4, wherein
the first elastic return member is a spring.

9. The hunchback orthosis as claimed in claim 3, wherein
each of the two first adjusting devices is an adjusting buckle, a hook and loop fastener, a snap or a magnetic connector.

10. The hunchback orthosis as claimed in claim 3, wherein
each of the two second adjusting devices is a hook and loop fastener, an adjusting buckle, a snap or a magnetic connector.
